(19) United States
(12) Patent Application Publication
(10) Pub. No.: US 2024/0177845 A1
(43) Pub. Date: May 30, 2024

(54) SYSTEM AND METHOD FOR SUPPLYING AND PURCHASING DURABLE MEDICAL EQUIPMENT

(71) Applicants: Adam Nadler, Parkland, FL (US); Philip Vasta, Boca Raton, FL (US); Adam Handfinger, Parkland, FL (US)

(72) Inventors: Adam Nadler, Parkland, FL (US); Philip Vasta, Boca Raton, FL (US); Adam Handfinger, Parkland, FL (US)

(21) Appl. No.: 18/523,520

(22) Filed: Nov. 29, 2023

Related U.S. Application Data
(60) Provisional application No. 63/428,457, filed on Nov. 29, 2022.

(51) Int. Cl. G16H 40/20; G06Q 40/08; G16H 10/60; G16H 15/00; G16H 20/10

(52) U.S. Cl. CPC G16H 40/20; G06Q 40/08; G16H 10/60; G16H 15/00; G16H 20/10; G16H 50/20

(57) ABSTRACT

A system for supplying and purchasing durable medical equipment, including at least one user computing device in operable connection with a network. An application server is in operable communication with the network to host an application program for supplying and purchasing durable medical equipment. The application program includes a user interface module for providing access to an inbox module, an intake module and onboarding module, and a medical record and notes review module the user interface module each provided on the user interface.

### CROSS-REFERENCE TO RELATED APPLICATIONS

The present application claims priority to U.S. Provisional Application No. 63/428,457 filed Nov. 29, 2022, titled “SOFTWARE AS A SERVICE PLATFORM FOR SUPPLYING AND PURCHASING DURABLE MEDICAL EQUIPMENT” which is hereby incorporated by reference in its entirety.

### TECHNICAL FIELD

The embodiments generally relate to the field of compliance and payment systems for home medical equipment, durable medical equipment. prosthetics, orthotics, and other supplies (DME or DMEPOS).

### BACKGROUND

The DME industry must abide by very strict regulations and meet numerous criteria in order to receive payment on a claim as well as to receive a DME license and accreditation. Once accredited and licensed a DME supplier can begin to bill Medicare for the supplies they provide to patients.

DME items may include, among many other things, oxygen and respiratory supplies, continuous positive airway pressure (CPAP machines), catheters, wheelchairs, canes, crutches, bath commodes, diabetic supplies, lancets, blood glucose meters, and continuous glucose monitors (CGMs).

In order for these items to be reimbursed by a Medicare program to a DME supplier, they must meet what is called Local Coverage Determination (LCD). LCDs are decisions made by a Medicare Administrative Contractor (MAC) whether to cover a particular item or service in a MAC's jurisdiction or region. MACs are Medicare contractors that develop LCDs and process Medicare claims.

Most DME suppliers may reach patients via direct-to-consumer marketing or direct to doctor referrals. DME suppliers may build a relationship with physicians and the physicians send DME suppliers' prescriptions for patients who need DME.

In order to fulfill the order for either scenario listed above, a process called medical review involves a DME supplier needing a signed prescription from the physician along with the patient's medical records to ensure that the supplies we are sending the patient meet the LCD in order for the DME supplier to get reimbursed.

DME suppliers all have to do this to ensure that each claim meets the medical necessity and LCD of the DME. Medicare routinely conducts audits, after orders for supplies have been fulfilled—and sometime even after claims for reimbursement have been paid and will randomly request documentation from DME suppliers. The documentation is submitted and reviewed and if the claim does not meet the LCD for the items furnished to the Medicare beneficiary, then the money paid to the DME supplier will be recouped along with potentially more significant consequences. Even if the supplies were sent to the Medicare beneficiary and the doctor signed off on the prescription it is the DME supplier's responsibility to review the medical records of the patient before sending the patient the product. If the medical records do not meet the LCD, the DME supplier can go back to the physician and request additional documentation or an addendum to the notes so the claim can be submitted properly and hold up against a future audit. This is a very big risk for DME suppliers as ensure compliance is complicated and the failure to have the appropriate documentation is very expensive—especially since the consequences of same typically occur after supplies have been provided to the patients and cannot be returned or re-sold. There are also potential criminal penalties for the lack of compliance.

In order to reduce waste and abuse in the industry, the Comprehensive Error Rate Testing (CERT) program evaluates a stratified random sample of Medicare FFS claims to determine if they were paid or denied properly under Medicare coverage, coding, and billing rules. The CERT program considers any payment for a claim that should have been denied or that was made in the wrong amount to be an improper payment. The findings can be projected to the entire universe of Medicare FFS claims because the CERT program ensures a statistically valid random sample. Therefore, the improper payment rate calculated from this sample is reflective of all claims processed by the Medicare FFS program during the report period.

In order for a DME supplier to operate and pass audits, they must enact a strict compliance program and also train their staff thoroughly on the Medicare coverage, Medicare Advantage, and commercial insurance company criteria and LCDs for the products they furnish to the patient. An audit can force a supplier out of business if not handled properly and can be costly to fight. But, even the strictest and most robust compliance programs are only as good as the individuals gathering and reviewing the appropriate documents. Mistakes are, unfortunately, often made.

It is the DME supplier's sole responsibility to collect documentation from the doctor's office. Despite the advance in technology, doctor's offices still use the fax machine as their main method of sharing information. Medical record keeping software is often proprietary and won't communicate with software from other hospitals or medical centers. Fax represents a simple workaround that's secure enough to meet patient privacy standards. The fax is considered more secure than email and cloud file sharing because it is less exposed. But the industry's insistence on using this antiquated technology further exacerbates DME suppliers' challenge of gathering, organizing, maintaining and reviewing these critical documents and the information contained therein.

Doctors have been faxing medical records long before the move to electronic health records. Electronic billing systems are too disjointed and not compatible with each other. The fax machine gets the job done regardless of what electronic billing system a hospital or clinic is using but has created a challenge for many reasons.

The use of the fax creates a challenge for the DME supplier and increases the workload significantly. The more patients the DME company is supplying items to the more human capital is required and this can be burdensome to an industry that has very tight margins.

A DME supplier often finds themselves fighting to get the medical records for the patient that the doctor provided the prescription for. Although Medicare has regulations allowing physicians to share documentation, a DME supplier often finds themselves in difficult situations trying to convince an office manager or a nurse to fax over the patient's progress notes. Much of the time it is never sent. This puts a lot of risk on the supplier if they are ever audited and do not have the proper documentation in the patient's file. Unfortunately orders for supplies are often fulfilled without the doctor's offices sending the appropriate paperwork, or the paperwork is lost, in very large part due to the logistical and technological challenges discussed herein.

A DME supplier is also faced with a tremendous amount of manual labor. There has not been much technological advancement in the DME space. Besides faxing, most of the work is done through spreadsheets and manual data entry. There has been very little attempt to introduce automation or any other technology to improve workflow.

On the occasions that the DME supplier does receive the documentation, it is often wrong. The physician does not know the LCD requirements for the supplies they are prescribing the patient. A physician wants the patient to receive it, a physician writes a prescription, and the physician expects the patient to get the supplies. This review process of the doctors' progress notes (when received) is done by the staff members of the DME company. Some of them have clinical experience such as registered nurses, medical assistants, and even pharmacy technicians. They are the ones that read through the notes, page by page and must decipher and locate where each of the points of the LCD are met. Not only is this a cumbersome process, but it is also ripe for human error. There is currently no technology system that helps DME suppliers gather, organize, maintain and review the documentation received from doctors to confirm that the points of the LCD are met.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The disclosed software as a service platform for supplying and purchasing durable medical equipment was developed to solve the described industry specific issues from both the DME suppliers' perspective, to protect them against any audits and recoupments, and Medicare's perspective, to avoid paying out unnecessary dollars for claims that do not meet the LCD criteria. Both sides of the transaction benefit by only fulfilling supply orders that are appropriate and compliant. The described platform can also be used by Medicare and Private insurance payers to confirm compliance before paying suppliers and satisfying claims for reimbursement. Private insurance payers and Medicare/Medicaid programs can implement this technology on their end prior to paying a claim because they do not have to hire and train people to manually do this review anymore. Rather than being reactive, insurance companies and payers (Medicare/Medicaid, and commercial insurance payers such as United Health Care, Aetna, Cigna, Humana and many more) can be proactive because they will have the tools they need to review claims in seconds rather than hours or days. This not only prevents waste to the insurer/payer but also can serve as a benefit to the DMEs that implement this technology in their workflow. Insurance companies will prevent loss and payments can be accelerated to the DME suppliers that use the software.

The system saves valuable time for medical supply companies and healthcare professionals by automating the addendum creation process. This enables them to focus more on their core responsibilities rather than administrative tasks. The system also reduces human error and mistakes.

The AI-driven platform minimizes errors and ensures that the necessary coverage criteria are accurately documented, reducing the risk of claim denials and facilitating faster reimbursement.

The system uses a HIPAA-compliant, password-protected email system for the secure transmission of sensitive medical information, ensuring patient privacy and adherence to industry standards.

The system also fosters seamless communication between medical supply companies and treating practitioners, eliminating the need for cumbersome document exchanges and expediting the addendum creation process.

By ensuring the medical records meet the required coverage criteria, the system helps protect medical supply companies from potential audits, safeguarding their financial interested and reputation. The System also provides the Medical Reviewer the exact criteria which must be me for each product and each payer, as the specific criteria can differ significantly, which is another cause of mistakes being made by the Medical Reviewers.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the computer system architecture, according to some embodiments;

FIG. 2 illustrates a screenshot of the inbox module and associated user interface, according to some embodiments;

FIG. 3 illustrates a screenshot of the intake module and onboarding module and associated user interface, according to some embodiments;

FIG. 4 illustrates a screenshot of the intake module and onboarding module and associated user interface, according to some embodiments;

FIG. 5 illustrates a screenshot of the intake module and onboarding module and associated user interface, according to some embodiments;

FIG. 6 illustrates a screenshot of the medical record and notes review module and associated user interface, according to some embodiments;

FIG. 7 illustrates a screenshot of the medical record and notes review module and associated user interface, according to some embodiments;

FIG. 8 illustrates a screenshot of the medical record and notes review module and associated user interface, according to some embodiments;

FIG. 9 illustrates a screenshot of the medical record and notes review module and associated user interface, according to some embodiments;

FIG. 10 illustrates a screenshot of the medical record and notes review module and associated user interface, according to some embodiments;

FIG. 11 illustrates a screenshot of the medical record and notes review module and associated user interface, according to some embodiments;

FIG. 12 illustrates a screenshot of the medical record and notes review module and associated user interface, according to some embodiments;

FIG. 13 illustrates a block diagram of the application program and computing system, according to some embodiments;

FIG. 14 illustrates a flowchart of a process for supplying and purchasing durable medical equipment, according to some embodiments;

FIG. 15 illustrates a screenshot of the resupply interface, according to some embodiments;

FIG. 16 illustrates a screenshot of the progress note request interface, according to some embodiments;

FIG. 17 illustrates a screenshot of the physician communication interface, according to some embodiments;

FIG. 18 illustrates a screenshot of the physician questionnaire interface, according to some embodiments;

FIG. 19 illustrates a screenshot of the progress notes interface, according to some embodiments; and

FIG. 20 illustrates a screenshot of the patient and physician information interface, according to some embodiments.

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, apparatus, software application for a mobile device, or computer program product at any possible technical detail level of integration. A computer application product can include, among other things, a computer-readable storage medium having computer-readable program instructions thereon for causing a processor to carry out aspects of the present disclosure.

As used herein, the term “user(s)” may refer to individuals accessing the system to develop, release, and promote media such as music.

As used herein, “GUI” may refer to any graphical user interface that includes at least one interactive component between a user and the application. A GUI may include a plurality of fillable fields, clickable buttons, database displays, or the like. A GUI maybe adaptable for use on several devices such as computers, phones, smart devices, tablets, laptops, televisions, or the like.

In general, the embodiments provided herein relate to a system and method for supplying and purchasing durable medical equipment. The system provides the means of receiving at least one of a facsimile or electronic communication and identifying a communication type.

In some embodiments, the system identifies a product category. For example, this product category can be continuous glucose monitors (or other category of medical devices). The medical record reviewer notes that the progress note needs to be analyzed against a series of machine learning modules or “smart tags” which were trained based on the LCD coverage criteria of the category of product that needs to be analyzed by the system.

In some embodiments, the system receives either a fax or electronic set of progress notes from the physician's office (which may be collectively referred to as the communication). The communication or progress note is then process by Optical Character Recognition (OCR) and electronically scanned for at least one document data relevant to the communication type. At least one previously trained machine learning module is called upon to correspond the communication type to known communication types. The system then verifies that the at least one of a facsimile or electronic communication contains information specific to the product category trained inside the platform.

In some embodiments, information is then correlated against the trained smart tags or algorithms from our system and the analysis is completed and a report can be generated in the form of a table of contents highlighting where each criteria can be found on the corresponding page.

In some embodiments, the system then automatically suggests if a communication complies and provides a method for the reviewer to confirm or disagree with the communication. User feedback is then provided and utilized to improve the accuracy of the back-end algorithms. Compliance of the information is verified within the communication to the communication type and the verified information is displayed. Non-verified information may also be displayed using a systematically generated addendum.

As used herein, the term “addendum” is used to describe an additional piece of information added to a medical record after the initial record has been created. An addendum is essentially a note or a clarification that provides additional details or corrects existing information. In the case of the system, using addendum intelligence, the system can be utilized to ask a doctor to please mention the diagnosis code for the patient because it was not mentioned, and it cannot be found in the medical record after our AI reviewed it.

For Medicare claims, especially for DME, it's vital that the medical records clearly support the necessity and criteria for the equipment prescribed. If the original medical record is lacking specific details required by Medicare (or other private payers like Humana, Aet, Cigna, etc.), an addendum can supply this missing information and complete the claim so it can be considered compliant and payable.

Medicare has stringent requirements on what is covered and why. If the medical record does not explicitly state the need and justification for the DME, the claim can be denied. An addendum helps ensure that all necessary details and information are included to meet the coverage criteria.

Reviewers can communicate and collaborate internally with their managers when reviewing the communication to confirm compliance. Managers of the reviewers (reviewers of potential mistakes) are automatically alerted, and the addendum is communicated electronically to an original facsimile or electronic communication sender. Data pertaining to the non-verified information is received and the patient report is updated.

At its core, the function the system includes the ability to utilize AI and Machine learning to read medical records and progress notes. The system recognizes what is missing from the medical records that is required by the insurance company, (e.g., Medicare, Medicaid, Commercial insurance payers, Medicare advantage payers, etc.). This is known in the industry as the coverage criteria and in Medicare language it is known as the LCD, (Local Coverage Determination).

If our system recognizes all the coverage criteria for a specific item is met, the user confirms within our UI and the claim can be processed. If something is missing from the medical records this is where our platform recognizes what is missing and proceeds with requesting the additional documentation and asking the physician's office for what is known as an addendum to the medical record.

In some embodiments, the system provides a transformative solution designed specifically for the dynamic and demanding Durable Medical Equipment (DME) and Home Medical Equipment (HME) industry. This solution aims to automate and improve the process of creating addendums, which are essential for ensuring accurate and comprehensive documentation in claim submission and reimbursements.

The process described herein works by utilizing artificial intelligence and machine learning to analyze medical records. It identifies missing coverage criteria automatically and subsequently generates a tailored, HIPAA-compliant questionnaire, specifically for what has been recognized by the system as missing from the medical records. This is known as what we called Addendum Intelligence (Trademarked). This questionnaire is sent securely to the treating practitioner via a password-protected email. Once the responses are received from the physician, Addendum Intelligence™ crafts a comprehensive progress note addendum that satisfies the required coverage criteria, facilitating successful claim submissions and payment processing.

In some embodiments, the system may best cater to doctor's offices that prefer using fax machines as their method of document exchange and communication. The system has the capability to feature to be used via fax communication and transmission. This is what we call the “analog” version. However, the system may offer both a digital version and analog version to accommodate different preferences and technological capabilities within the medical community.

The digital embodiments may utilize advanced AI and machine learning to analyze medical records, identify gaps in coverage criteria, and create tailored, HIPAA-compliant questionnaires. These questionnaires are then sent digitally to the treating practitioners for completion, with the responses used to craft comprehensive progress note addendums. This digital version is ideal for healthcare providers who are comfortable with and capable of managing electronic communications and records.

The analog embodiments cater to healthcare providers who still rely on fax machines for their documentation and communication needs. In this scenario, the system can send out an addendum request via fax, receive it back via fax, analyze it to determine if any additional information is required and then communicate back with the healthcare provider again until the supplier has all the medical information they need to submit a claim. This version ensures that those who are not fully integrated into digital workflows can still benefit from the advanced capabilities of the platform.

By offering both digital and analog versions, Addendum Intelligence™ ensures a wide reach and usability across the DME industry, accommodating varying levels of technological adoption in different medical practices. This approach not only enhances operational efficiency but also ensures inclusivity, allowing all types of healthcare providers to benefit from this innovative solution.

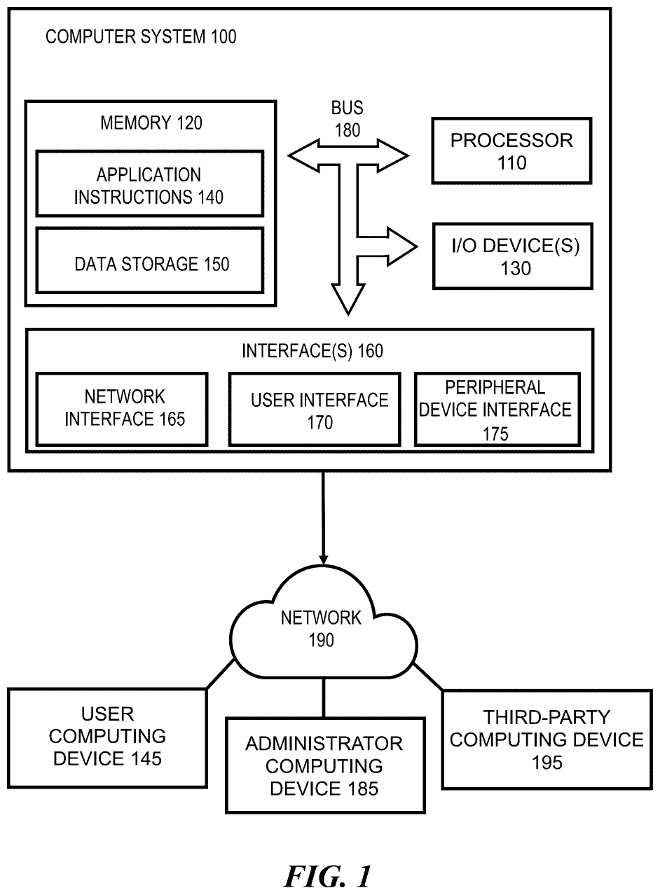

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

The disclosed system combines e-prescriptions with artificial intelligence and machine learning to ensure claims sent to Medicare and other insurance payers meet the necessary coverage criteria, LCDs, for each product in order to receive reimbursement.

The disclosed system may be trained on algorithms related to certain LCDs so when progress notes are received from a doctor's office, regardless of delivery method, they are uploaded into the system and analyzed by artificial intelligence.

The disclosed system may be implemented on the DME supplier side and may be constructed and arranged to receive a fax or electronic document for a prescription, analyze the fax or electronic document and automatically extract the data to create a patient record. From there, depending on what type of progress note received, a staff member selects the progress note type (for example Continuous Glucose Monitor) and the algorithms trained specifically for the CGM LCDs are called and reviewed by the AI.

The system may display all of the data within a user interface with either a light green check representing the item was found or a light red “x” indicating the system did not find details in the progress note. The human Medical Reviewer must then confirm or reject the recommendation and once it does the color will change to either dark red or dark green, depending upon the result of the review. If the response is different than that which was recommended by the platform a report is generated so that the work and judgment of the human Medical Reviewer can be confirmed and, if correct, a determination can be made whether any particular algorithms need to be retrained or refined in some way. The LCD items are highlighted in the user interface for reference and in the case of an audit can be referenced at any time and provided to Medicare or any other insurer requesting the documentation. This also saves time when a company responds to an audit. Rather than having to go through the documentation again, the system highlights each LCD requirement and also produces a summary of findings for each progress note reviewed that can be downloaded and packaged when responding to an audit. This makes it easy to see that coverage criteria has been met. The records with an index and the highlighted LCD items can be downloaded to an interactive PDF and stored in any platform.

If an item from the LCD is missing, the system may generate an addendum to electronically send back to the doctor with the necessary questions required to be answered to complete the progress note and submit a clean claim to Medicare. The system may update the original patient record. The physician can e-sign the document and answer only the necessary questions that were missing in order to submit the claim. Answers may be input into the system and the LCD may be updated. This results in the patient receiving their medical supplies faster and provides better quality of care.

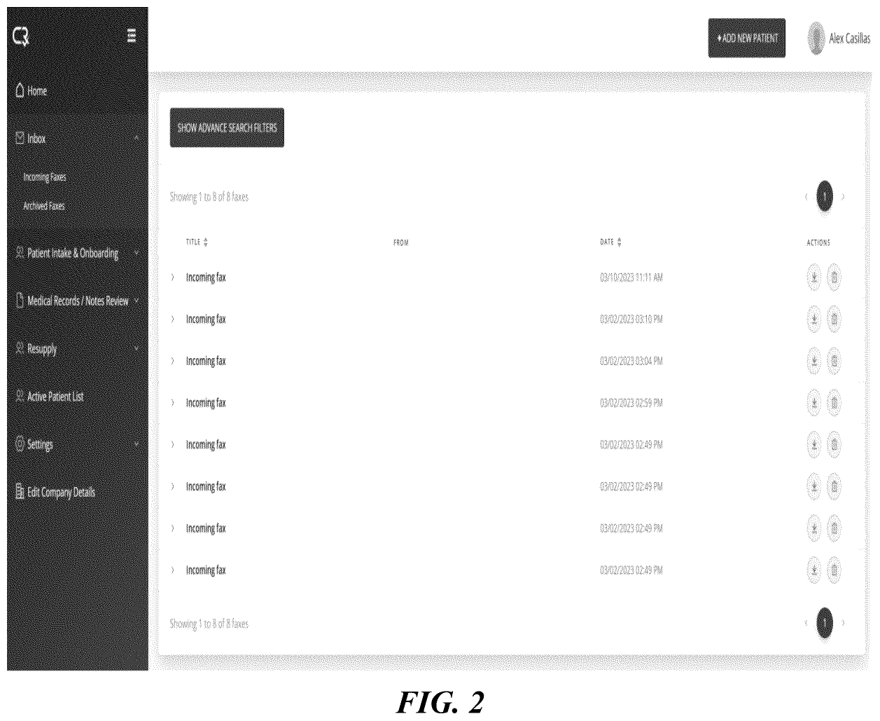

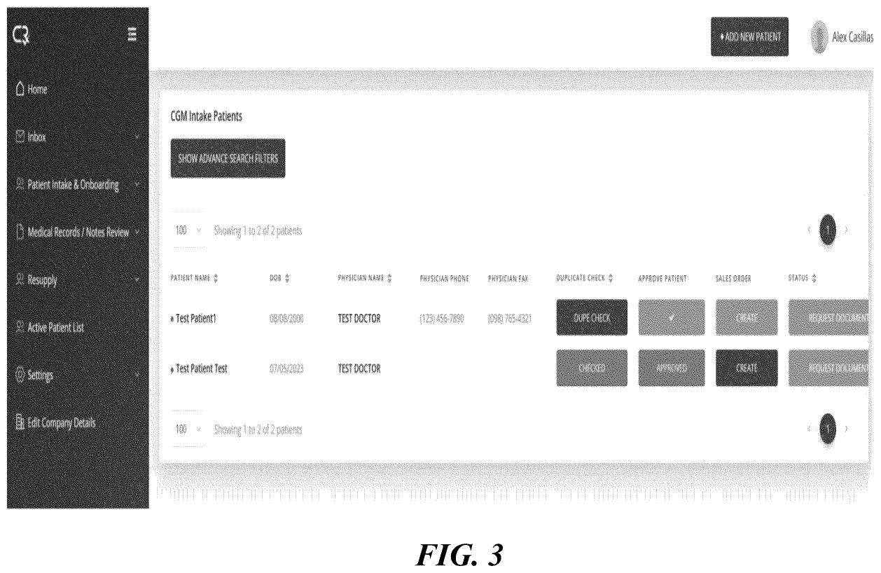

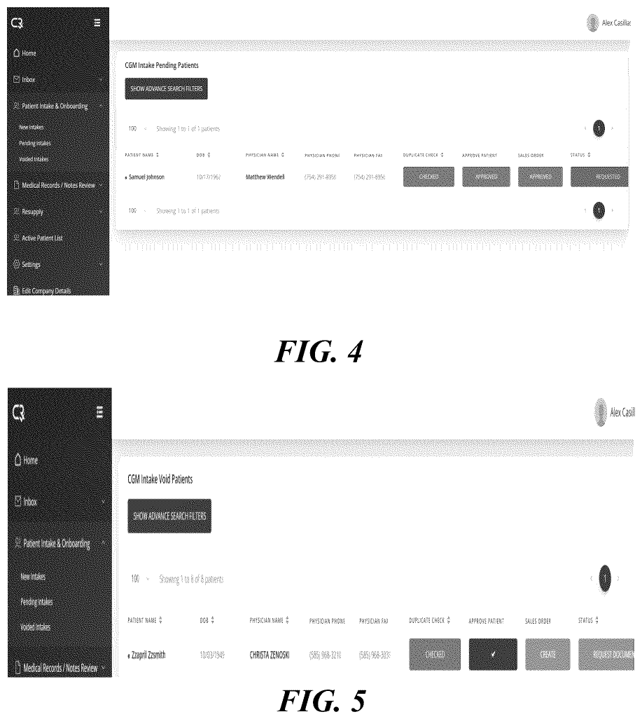

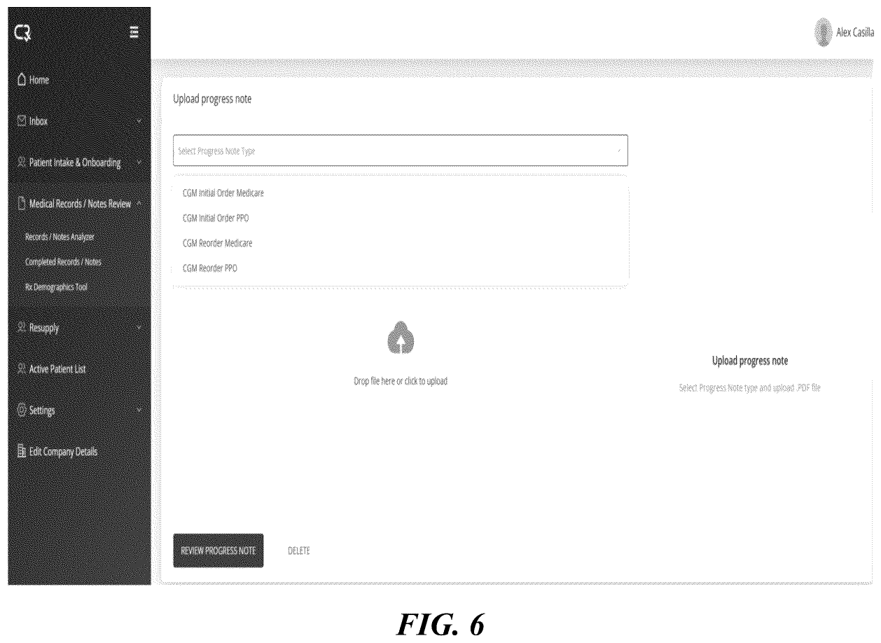

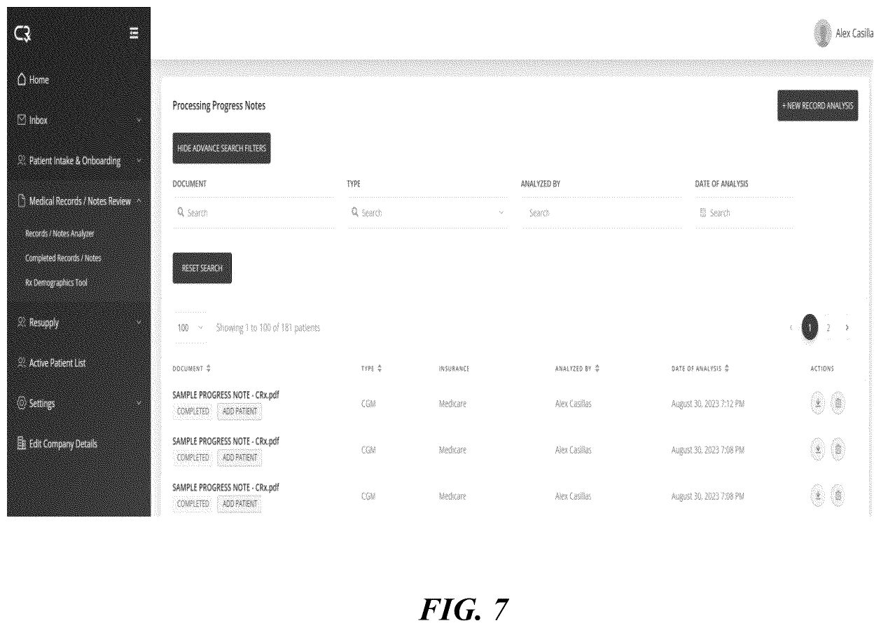

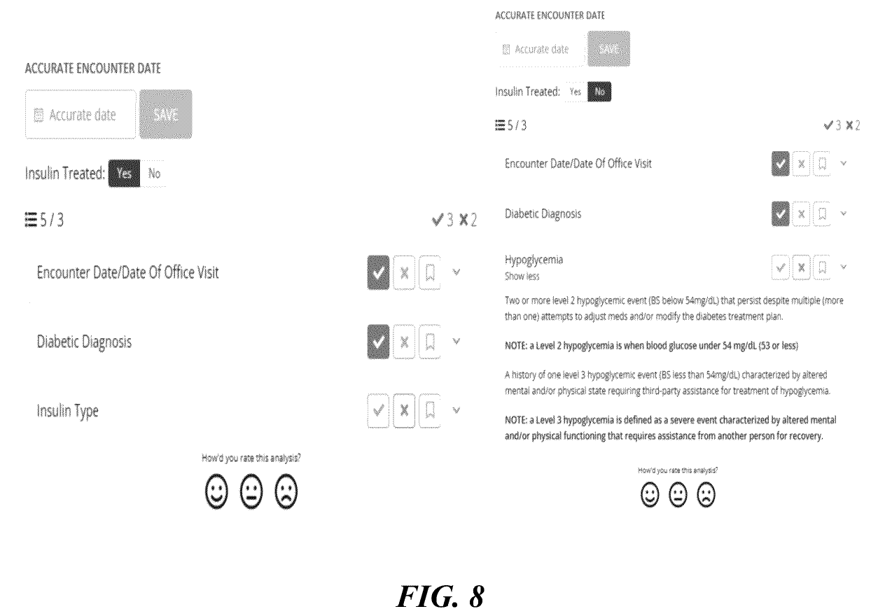

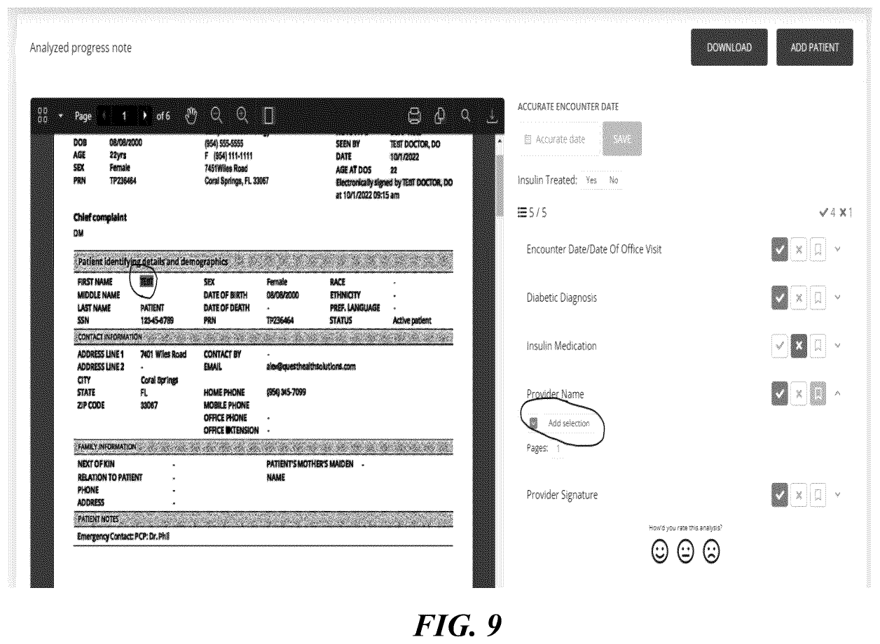

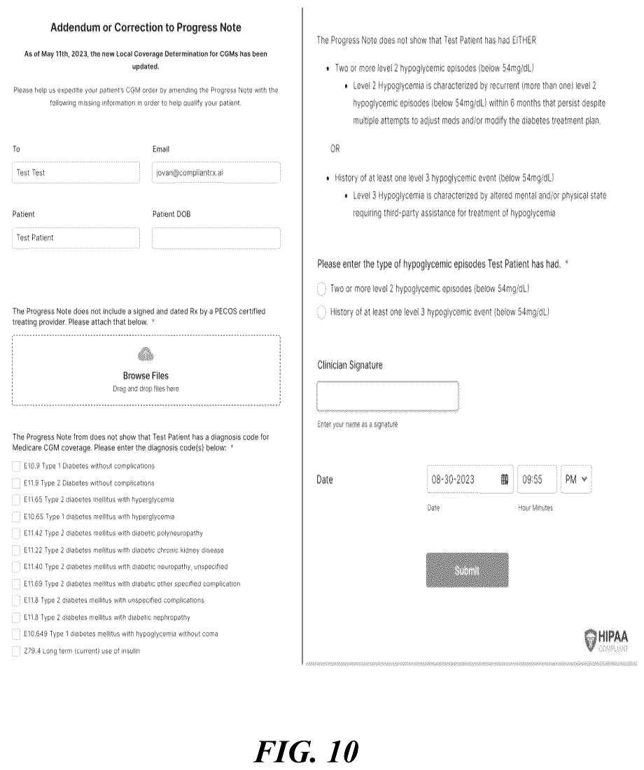

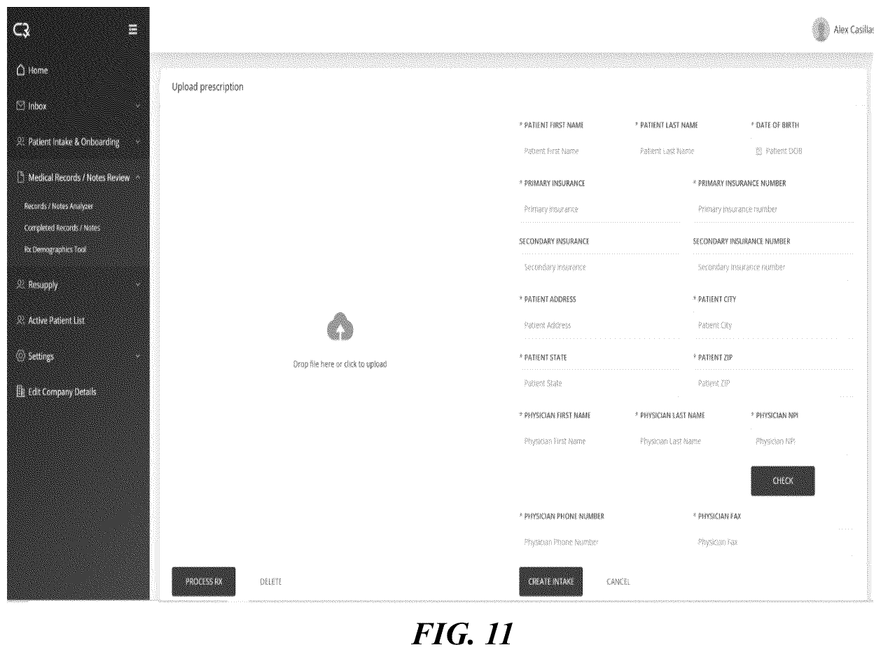

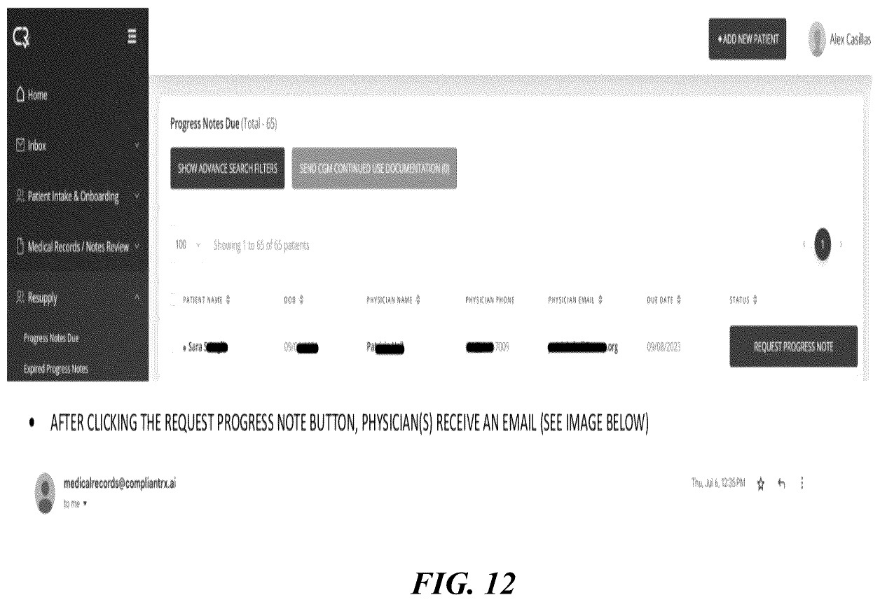

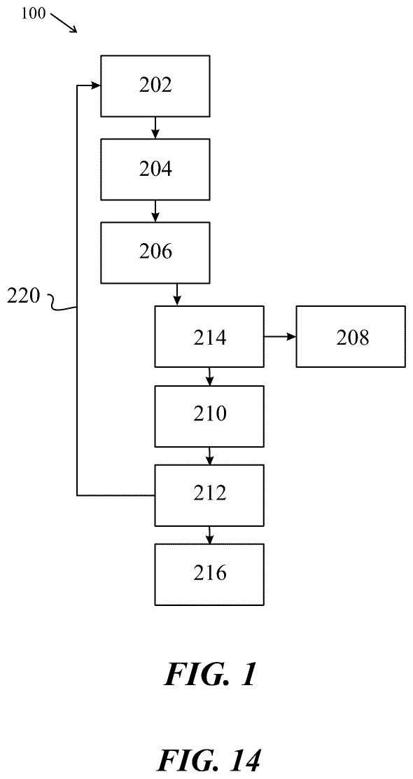

FIG. 2 illustrates a screenshot of the inbox module and associated user interface. FIGS. 3-5 illustrate screenshots of the intake module and onboarding module and associated user interface. FIGS. 6-12 illustrate screenshots of the medical record and notes review module and associated user interface.

To be eligible for coverage of a CGM and related supplies, the beneficiary must meet all of the following initial coverage criteria.
1. The beneficiary has diabetes mellitus (Refer to the ICD-10 code list in the LCD-related Policy Article for applicable diagnoses); and,
2. The beneficiary's treating practitioner has concluded that the beneficiary (or beneficiary's caregiver) has sufficient training using the CGM prescribed as evidenced by providing a prescription; and,
3. The CGM is prescribed in accordance with its FDA indications for use; and,
4. The beneficiary for whom a CGM is being prescribed, to improve glycemic control, meets at least one of the criteria below:
   a. The beneficiary is insulin-treated with at least one daily administration of insulin; or,
   b. The beneficiary has a history of problematic hypoglycemia with documentation of at least one of the following:
      i. Recurrent level 2 hypoglycemic events (glucose <54 mg/dL (3.0 mmol/L) that persist despite multiple (2 or more) attempts to adjust medication(s) and/or modify the diabetes treatment plan; or
      ii. A history of one level 3 hypoglycemic event (glucose <54 mg/dL (3.0 mmol/L) characterized by altered mental and/or physical state requiring third-party assistance for treatment of hypoglycemia
5. Within six (6) months prior to ordering the CGM, the treating practitioner has an in-person or Medicare-approved telehealth visit with the beneficiary to evaluate their diabetes control and determined that criteria (1-4) above are met.

Every six months following the initial prescription of the CGM, the treating practitioner conducts an in-person or Medicare-approved telehealth visit with the beneficiary to document adherence to their CGM regimen and diabetes treatment plan. Looking at this requirement above, a patient must see a doctor either face-to-face or through a Medicare approved telehealth visit.

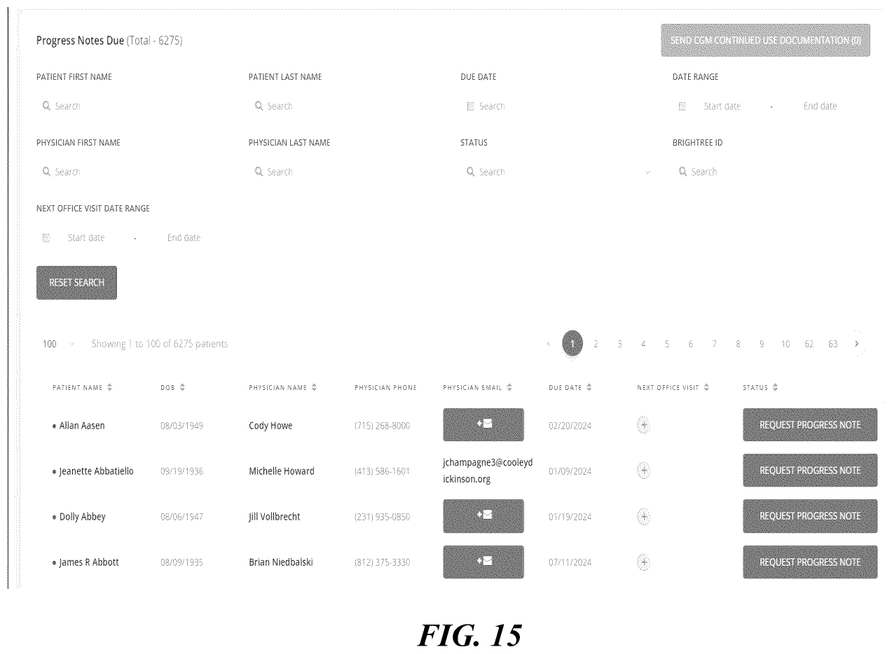

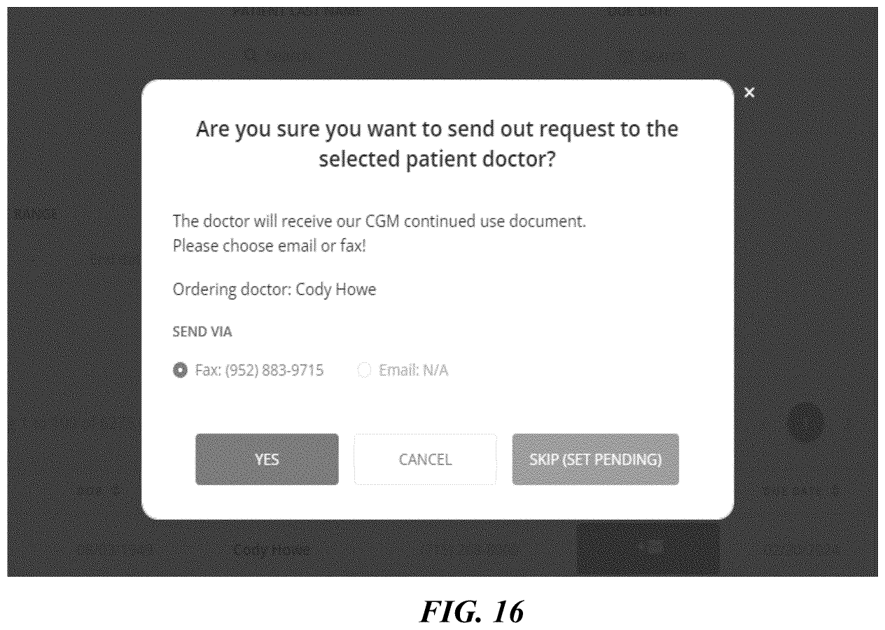

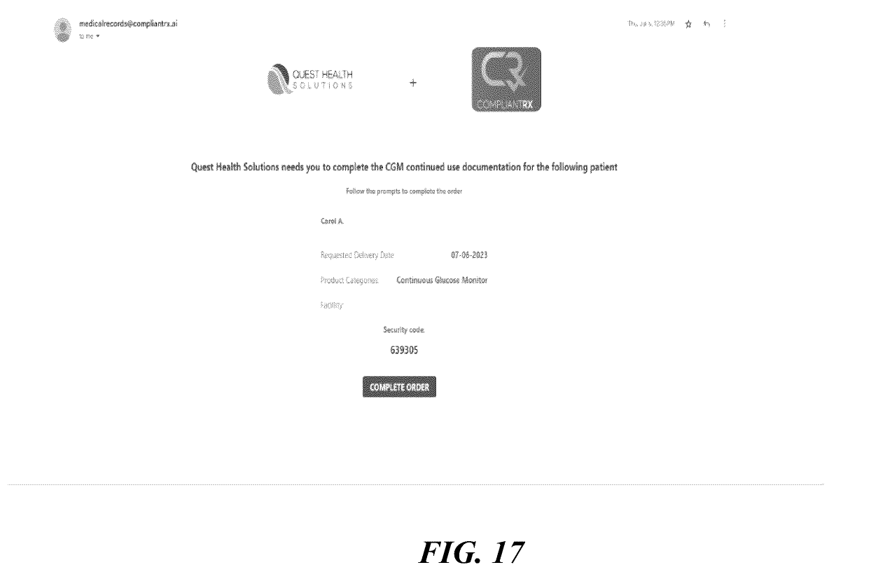

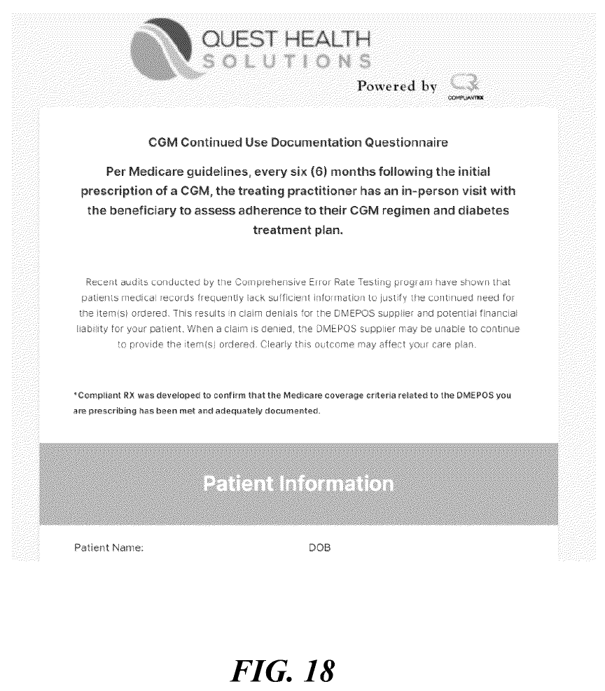

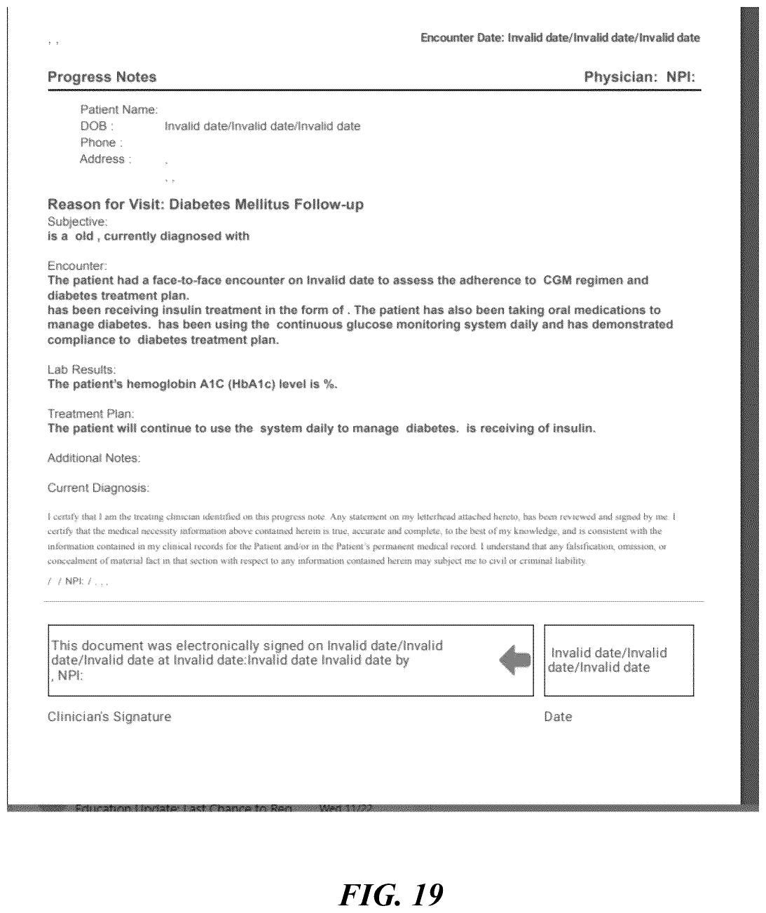

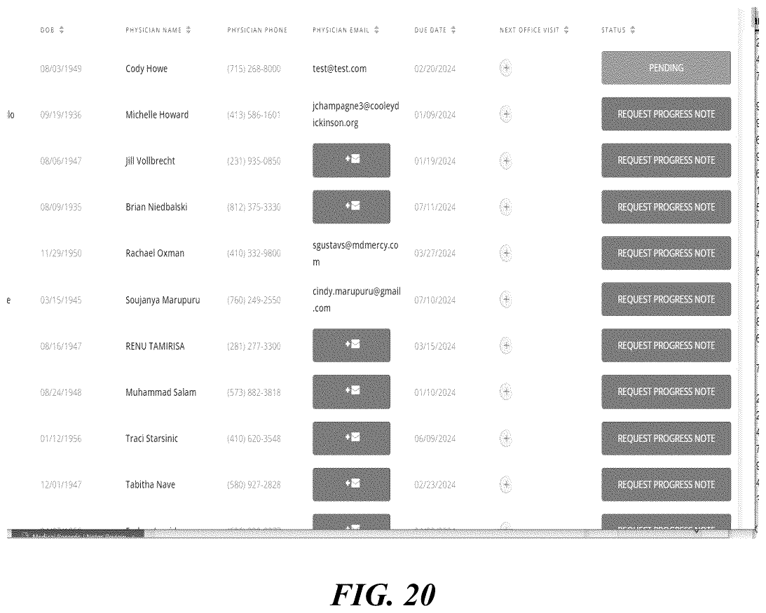

The challenge remains the same, the physician has to put in her notes that the patient is using a CGM and adhering to their diabetic treatment plan. Inside of the platform, an entire workflow has been developed surrounding this requirement through e-prescription capability and automation. FIGS. 15-20 illustrate screenshots of the process that automates this if a physician has email and electronic communication. If they do not, we still request the medical records from the last visit via fax which is then returned to the patient's account, read by our AI system and confirms if the requirements for continued use are met. FIGS. 15-20 are further described below. The system is able to automatically track when a patients six month time is coming up and request the notes from the physician prior to the due date. With one click of “request progress note”, the order can be sent to physician directly via email if it is on file or fax. If sent by fax it is traditional fax cover sheet, but if sent by email this is where the platform is special. Once yes is selected, the physician logs in and may fill out a questionnaire pertaining to the patient.

The final outcome after the physician answers all of the pertinent questions about diabetes treatment adherence and using the CGM our platform creates a Progress note that DME suppliers can add to the patient's account and use that as the compliant documentation to continue servicing the patient. At the same time, the doctor will get a copy of this progress note and be able to add it to the patient record.

By making this process completely electronic through an e-prescribed questionnaire that results in a progress note it avoids the back and forth between faxing medical records back and forth to the DME supplier. The CGM continued use documentation questionnaire can only be submitted if the answers are answered correctly therefore avoiding the need to review the information and request additional documentation. Everything the DME supplier needs to continue servicing the patient is now complete upon receipt saving a tremendous amount of time and staff work manually reviewing additional documentation and requesting more information if it is not in the initial notes. This can streamline the process of managing many patients and keeping track of who is coming up due for note requalification with the due date feature and our customizable search feature.

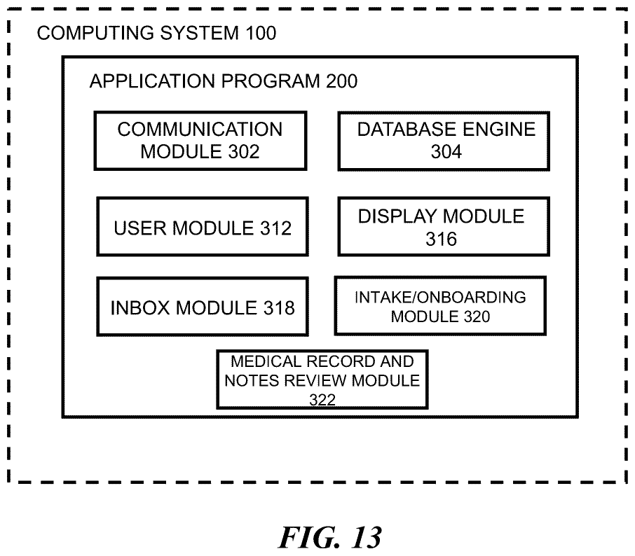

Referring to FIG. 13, the system 200 may receive 202 a facsimile or electronic communication, such as, but not limited to, a prescription for a healthcare provider. The system may scan 204 the communication for relevant document information. According to some embodiments, a system user may input a communication type, such as a prescription for a specific DME, such as a CGM, CPAP device, ostomy bag, oxygen, respiratory supplies etc., such that the system associates a communication type with the communication. The system may subsequently call machine learning module(s) previously trained corresponding to the specific communication type and verify that the communication contains all required information specific to the communication type. The system may generate a patient report 206 within the system correlating information within the communication to the specific communication type. Similarly, the system may verify compliance 214 of the information, such that the information meets coverage criteria, within the communication to the identified communication type and display 208 verified information. Non-verified information may be systematically flagged as such and displayed 208 to an end user. The system may also systematically generate an addendum 210 identifying non-verified information and requiring 212 non-verified information from the original communication sender. The addendum 210 may be communicated electronically 220 to the original communication sender who may provide the non-verified information which may be received 202 by the system. In this way, the system may verify that all relevant information is present within the patient report depending on the communication type. The system may then proceed to billing 216.

FIG. 14 illustrates an example computer architecture for the application program 200 operated via the computing system 100. The computing system 100 comprises several modules and engines configured to execute the functionalities of the application program 200, and a database engine 204 configured to facilitate how data is stored and managed in one or more databases. In particular, FIG. 14 is a block diagram showing the modules and engines needed to perform specific tasks within the application program 200.

Referring to FIG. 14, the computing system 100 operating the application program 200 comprises one or more modules having the necessary routines and data structures for performing specific tasks, and one or more engines configured to determine how the platform manages and manipulates data. In some embodiments, the application program 300 comprises one or more of a communication module 302, a database engine 304, a user module 312, a display module 316, an inbox module 318, and an intake module and onboarding module 320, and a medical record and notes review module 322.

In some embodiments, the communication module 302 is configured for receiving, processing, and transmitting a user command and/or one or more data streams. In such embodiments, the communication module 302 performs communication functions between various devices, including the user computing device 145, the administrator computing device 185, and a third-party computing device 195. In some embodiments, the communication module 302 is configured to allow one or more users of the system, including a third-party, to communicate with one another. In some embodiments, the communications module 302 is configured to maintain one or more communication sessions with one or more servers, the administrative computing device 185, and/or one or more third-party computing device(s) 195.

In some embodiments, the communication module 302 allows for the transmission patient information, insurance provider information, third-party information, and the like. This information is used by the system in the various ways described hereinabove.

In some embodiments, a database engine 304 is configured to facilitate the storage, management, and retrieval of data to and from one or more storage mediums, such as the one or more internal databases described herein. In some embodiments, the database engine 304 is coupled to an external storage system. In some embodiments, the database engine 304 is configured to apply changes to one or more databases. In some embodiments, the database engine 304 comprises a search engine component for searching through thousands of data sources stored in different locations.

The user module 312 may store user preferences including the user account information, historical usage data, user personal information, patient information, third party information and the like.

In some embodiments, the display module 316 is configured to display one or more graphic user interfaces, including, e.g., one or more user interfaces, one or more consumer interfaces, one or more video presenter interfaces, etc. In some embodiments, the display module 316 is configured to temporarily generate and display various pieces of information in response to one or more commands or operations. The various pieces of information or data generated and displayed may be transiently generated and displayed, and the displayed content in the display module 316 may be refreshed and replaced with different content upon the receipt of different commands or operations in some embodiments. In such embodiments, the various pieces of information generated and displayed in a display module 316 may not be persistently stored. The display module 316 provides alerts to the user device which can be viewed and acknowledged by the user.

In some embodiments, the inbox module 318 wherein faxes may be received in the inbox via a dedicated fax number. The user may opt to download fax files and/or delete files using the inbox module. Similarly, other communication may be visualized, interacted with, created, and transmitted using the inbox module 318 and communication module 202.

In some embodiments, the intake module and onboarding module 320 is capable of capturing patient data from an external document (eFORM). The form data is transmitted into the intake and onboarding module 320 to allow for the patient to be approved, sales orders to be created and ordered, documents to be sent, requested, etc. When a patient's orders are fulfilled in the CRM, the patient account transitions from a pending intake account to an active patient account. When a patient's account is voided in the CRM, the account is removed.

In some embodiments, the medical record and notes review module 322 is capable of permitting the analysis of a medical record. This module may display the durable medical equipment type, order stage type, insurance payor, and other medical information. Progress notes may also be analyzed, and the user may receive a notification and the analyzed medical record is stored in the completed records/notes section of the medical record and notes review module 322. From the completed records/notes section, the user may search for completed records and begin a new record analysis. The user may also select a completed record to conduct a quality assurance review of the completed medical record. Criteria that are found within the analyzed document are highlighted within the document. Criteria that is missing within the analyzed document may be indicated on the interface, along with found criteria.

In some embodiments, the user may conduct a quality review by verifying any of the above information. If criteria exists and the analyzer excluded the criteria result from the analysis, users can manually annotate the interface. Once the quality review is completed, the user can add the completed record to a patient's account or create an account if needed. If any criteria is missing, the user can request an addendum from the physician.

In some embodiments, the user may upload, delete, or otherwise interact with a prescription document which is uploaded to the system.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

I/we claim:

1. A system for supplying and purchasing durable medical equipment, the system comprising:
at least one user computing device in operable connection with a network;
an application server in operable communication with the network, the application server configured to host an application program for supplying and purchasing durable medical equipment, the application program having a user interface module for providing access to an inbox module, an intake module and onboarding module, and a medical record and notes review module the user interface module each provided on the user interface.

2. The system of claim 1, wherein the inbox module displays one or more incoming fax documents.

3. The system of claim 1, wherein the intake module and onboarding module allows for the capture of patient data from an external eFORM.

4. The system of claim 1, wherein a patient account is defined as a pending intake account if the patient's orders are unfulfilled.

5. The system of claim 1, wherein a patient account is defined as an active intake account if the patient's orders are fulfilled.

6. The system of claim 1, wherein the medical record and notes review module permits the analysis of a medical record.

7. The system of claim 6, wherein the analysis of the medical record permits the selection of a medical record type and analysis option.

8. The system of claim 7, wherein the medical record and notes review module permits the completion of a quality assurance review via the user.

9. The system of claim 8, wherein the quality assurance review includes verifying a plurality of found criteria in the medical record.

10. The system of claim 9, wherein the quality assurance review includes verifying a plurality of missing criteria in the medical record.

11. The system of claim 10, further comprising an analyzer capable of determining if the plurality of missing criteria and the plurality of found criteria exist in the medical record.

12. The system of claim 11, wherein the medical record and notes review module permits the request for an addendum, wherein the request for the addendum is transmitted to a physician.

13. The system of claim 12, further comprising a prescription uploaded by the user, the prescription comprising a plurality of prescription information.

14. The system of claim 13, wherein a plurality of prescription information is extracted from the prescription.

15. The system of claim 14, wherein the user adds, deletes, or edits the plurality of extracted prescription information.

16. The system of claim 1, further comprising a database engine to store a plurality of patient information, a plurality of physician information, a plurality of prescription information, and a plurality of medical record information.

17. The system of claim 1, wherein compliance of the medical record information is verified within the communication.

18. The system of claim 1, further comprising communicating the addendum electronically to an original facsimile or an electronic communication sender.

19. A method comprising:
receiving at least one of a facsimile or electronic communication;
identifying a communication type;
electronically scanning the communication for at least one document data relevant to the communication type;
calling at least one machine learning module previously trained corresponding to the communication type;
verifying that the at least one of a facsimile or electronic communication contains information specific to the communication type;
correlating information within the communication to the communication type;
generating a patient report;
automatically suggesting if a communication complies and providing a method for the reviewer to confirm or disagree with same;
providing and utilizing user feedback to improve the accuracy of one or more back-end algorithms;
verifying compliance of the information within the communication to the communication type;
displaying at least one verified information;
displaying a non-verified information;
systematically generating an addendum identifying non-verified information;
providing a way for reviewers to communicate and collaborate internally with their managers when reviewing the communication to confirm compliance;
automatically alerting the managers of the reviewers of potential mistakes in the review process;
communicating the addendum electronically to an original facsimile or electronic communication sender;
receiving data pertaining to the non-verified information
updating the patient report.
